(19) United States
(12) Patent Application Publication
(10) Pub. No.: US 2021/0275144 A1
(43) Pub. Date: Sep. 9, 2021

(54) ULTRASONIC INSPECTION APPARATUS

(71) Applicant: MELODY INTERNATIONAL LTD., Kagawa (JP)

(72) Inventor: Yhuko OGATA, Kagawa (JP)

(73) Assignee: MELODY INTERNATIONAL LTD., Kagawa (JP)

(21) Appl. No.: 17/327,754

(22) Filed: May 23, 2021

Related U.S. Application Data
(63) Continuation of application No. PCT/JP2019/036218, filed on Sep. 13, 2019.

(30) Foreign Application Priority Data
Nov. 28, 2018 (JP) 2018-222107

(51) Int. Cl. A61B 8/00; A61B 8/08

(52) U.S. Cl. CPC A61B 8/46; A61B 8/462; A61B 8/488

(57) ABSTRACT

An ultrasonic inspection apparatus comprises: an ultrasonic sensor unit including an ultrasonic transmission unit for transmitting an ultrasonic wave and an ultrasonic reception unit for receiving a reflected ultrasonic wave, the reflected ultrasonic wave being an ultrasonic wave resulting from the transmitted ultrasonic wave transmitted by the ultrasonic transmission unit being reflected in a human body; a Doppler signal generation unit for generating a Doppler signal based on a difference between a frequency of the transmitted ultrasonic wave and a frequency of the reflected ultrasonic wave; a sound signal generation unit for generating a sound signal corresponding to the Doppler signal; and a speaker for outputting the sound signal.

### CROSS-REFERENCE TO RELATED APPLICATIONS

The present application is a continuation application of International Application No. PCT/JP2019/036218, filed on Sep. 13, 2019, which claims priority to Japanese Patent Application No. 2018-222107, filed on Nov. 28, 2018. The contents of these applications are incorporated herein by reference in their entirety.

### BACKGROUND OF THE INVENTION

The present invention relates to ultrasonic inspection apparatuses for inspecting human bodies using ultrasonic waves.

There are conventionally known apparatuses that are capable of identifying states in bodies by transmitting ultrasonic waves. Japanese Translation of PCT International Application Publication No. 2018-527101 discloses an ultrasonic inspection apparatus capable of identifying a fetal heart rate based on ultrasonic Doppler echo signals.

Conventional ultrasonic inspection apparatuses generate data for enabling visualization of state in a human body by processing ultrasonic Doppler echo signals generated on the basis of (i) ultrasonic waves emitted from an ultrasonic transducer disposed on a surface of the human body, and (ii) ultrasonic waves reflected within the body. The ultrasonic inspection apparatus may display the generated data on a monitor connected via a network.

The intensity of the ultrasonic waves reflected within the body varies depending on the position of the ultrasonic transducer. Therefore, for checking the state of a specific region, an inspector needs to move the ultrasonic transducer to a position suitable for the inspection of the region to be checked. The conventional ultrasonic inspection apparatuses, if used, suffer from the problem of poor operability in that the inspector needs to move an ultrasonic device while alternating between looking at the image displayed on the monitor connected via the network and looking at the human body to be inspected in order to find an optimum position.

### BRIEF SUMMARY OF THE INVENTION

The present invention has been made in view of these points, and an object of the present invention is to improve operability of an ultrasonic inspection apparatus.

An ultrasonic inspection apparatus of the present invention includes: an ultrasonic sensor unit including an ultrasonic transmission unit for transmitting an ultrasonic wave and an ultrasonic reception unit for receiving a reflected ultrasonic wave, the reflected ultrasonic wave being an ultrasonic wave resulting from the transmitted ultrasonic wave that is transmitted by the ultrasonic transmission unit and is reflected in a human body; a Doppler signal generation unit for generating a Doppler signal based on a difference between a frequency of the transmitted ultrasonic wave and a frequency of the reflected ultrasonic wave; a sound signal generation unit for generating a sound signal corresponding to the Doppler signal; and a speaker for outputting the sound signal.

### BRIEF DESCRIPTION OF THE DRAWINGS

FIG. 1A to 1C are diagrams showing an external appearance of an ultrasonic inspection apparatus 1.

FIG. 2 is a diagram showing a position where an ultrasonic sensor is disposed inside the ultrasonic inspection apparatus 1.

FIG. 3 is a schematic diagram showing an inner structure of the ultrasonic inspection apparatus 1.

FIG. 4 is a block diagram showing a functional configuration of the ultrasonic inspection apparatus 1.

FIG. 5 is a diagram showing a configuration example of a sound signal generation unit 252.

FIG. 6A to 6C are diagrams for explaining a multiplication process in the sound signal generation unit 252.

FIG. 7 is a diagram showing an external appearance of an ultrasonic inspection apparatus 2.

FIG. 8 is a block diagram showing a functional configuration of the ultrasonic inspection apparatus 2.

FIG. 9A to 9C are diagrams showing an external appearance of an ultrasonic inspection apparatus 2a.

FIG. 10 is a block diagram showing a functional configuration of an ultrasonic inspection apparatus 3.

### DETAILED DESCRIPTION OF THE INVENTION

Hereinafter, the invention will be described through embodiments of the invention. The below embodiments, however, are not intended to limit the invention according to the claims, and all combinations of features described in the embodiments are not necessarily essential to the solutions of the invention.

### First Embodiment

### [Outline of Ultrasonic Inspection Apparatus 1]

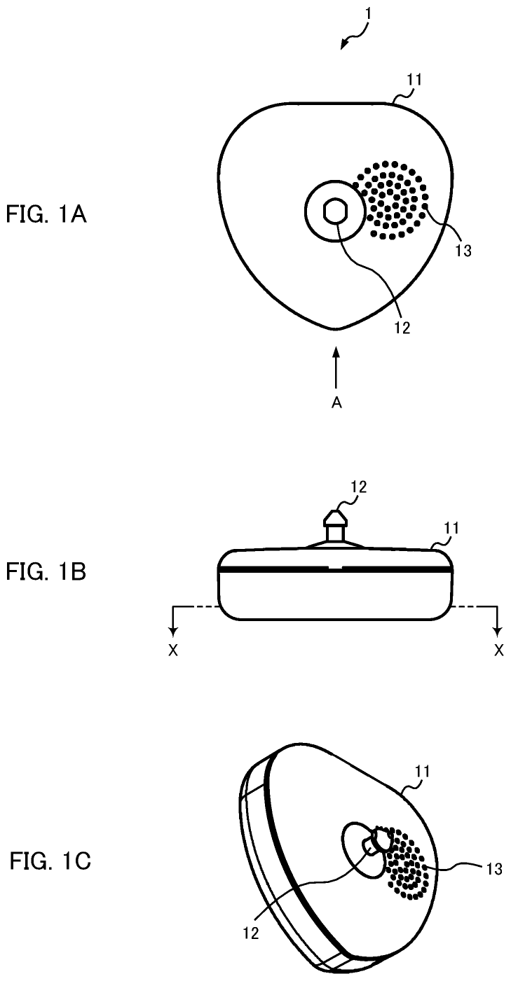

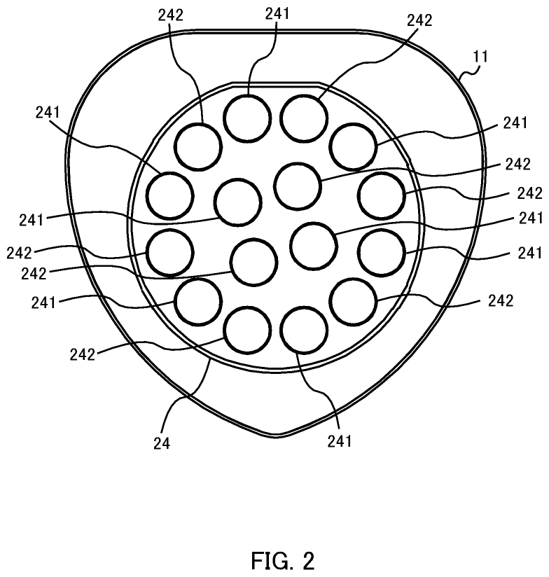

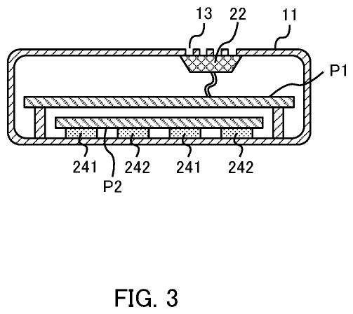

FIG. 1A to 1C are diagrams showing an external appearance of an ultrasonic inspection apparatus 1. FIG. 2 is a diagram showing a position where an ultrasonic sensor is disposed inside the ultrasonic inspection apparatus 1, and corresponds to a cross-sectional view taken along line X-X in FIG. 1B. FIG. 3 is a schematic diagram showing an inner structure of the ultrasonic inspection apparatus 1. FIG. 1A is a top view of the ultrasonic inspection apparatus 1 and FIG. 1B is a view of the ultrasonic inspection apparatus 1 when seen from the side with the arrow A in FIG. 1A. FIG. 1C is a perspective view of the ultrasonic inspection apparatus 1.

Hereinafter, the configuration of the ultrasonic inspection apparatus 1 will be outlined with reference to FIGS. 1A to 3. The ultrasonic inspection apparatus 1 includes a protrusion 12 provided on a housing 11 and an aperture 13 formed in the housing 11. The protrusion 12 is used, for example, for connecting a belt for fixing the ultrasonic inspection apparatus 1 at an optimum position by a person (hereinafter referred to as a “user”) who performs an inspection using the ultrasonic inspection apparatus 1.

The ultrasonic inspection apparatus 1 is an apparatus having an ultrasonic transducer for measuring, for example, a fetal heart rate. When performing an inspection, the user moves a surface of the ultrasonic inspection apparatus 1 on the side opposite to the side on which the protrusion 12 is provided while causing the surface thereof to contact the surface of the human body. The ultrasonic inspection apparatus 1 generates a signal corresponding to the cycle and intensity of the fetal heart rate on the basis of a Doppler signal that is based on a difference between the frequency of an ultrasonic wave transmitted from an ultrasonic transmission device provided inside the housing 11 (hereinafter referred to as a “transmitted ultrasonic wave”) and the frequency of a reflected ultrasonic wave, which is an ultrasonic wave resulting from the transmitted ultrasonic wave reflected within the human body.

The ultrasonic inspection apparatus 1 transmits the signal generated based on the Doppler signal to an external terminal via, for example, a wireless communication line. The external terminal may be, for example, a smartphone, a tablet, a computer, or a display. The user can visually confirm, for example, a waveform indicating the cycle and intensity of the heart rate as the signal based on the Doppler signal at the external terminal.

The ultrasonic inspection apparatus 1 also includes a speaker 22 therein, and the speaker emits a sound signal corresponding to the Doppler signal. The sound signal is emitted to the outside of the ultrasonic inspection apparatus 1 via the aperture 13 formed in the housing 11. When inspecting the fetal heart rate, the user can intuitively grasp the fetal state because the user can hear the fetal heart sound generated from the speaker.

The ultrasonic inspection apparatus 1 emits, for example, a sound signal having a magnitude corresponding to the intensity of the reflected ultrasonic wave or the intensity of the Doppler signal. The intensity of the reflected ultrasonic wave is represented, for example, by the maximum voltage of the reflected ultrasonic wave, and the intensity of the Doppler signal is represented, for example, by the maximum voltage of the Doppler signal. With this configuration of the ultrasonic inspection apparatus 1, the closer the ultrasonic inspection apparatus 1 is to the position of the fetal heart, the more loudly the user can hear the fetal heart sound. Therefore, the user can search for a position where the heart sound becomes larger while listening to the heart sound generated by the ultrasonic inspection apparatus 1, and it is therefore easy to move the ultrasonic inspection apparatus 1 to an appropriate position.

As shown in FIG. 2, the ultrasonic inspection apparatus 1 includes a plurality of ultrasonic transmission units 241, which are devices for transmitting ultrasonic waves, and a plurality of ultrasonic reception units 242, which are devices for receiving reflected ultrasonic waves. Provided in the example shown in FIG. 2 are six ultrasonic transmission units 241 and six ultrasonic reception units 242 arranged circumferentially, as well as two ultrasonic transmission units 241 and two ultrasonic reception units 242 arranged on the inward side of the ultrasonic transmission units 241 and the ultrasonic reception units 242 that are arranged circumferentially; however, the number of the ultrasonic transmission units 241 and the ultrasonic reception units 242 of the ultrasonic inspection apparatus 1 is arbitrary. Further, in the example shown in FIG. 2, the ultrasonic transmission units 241 and the ultrasonic reception units 242 are arranged in an alternating manner; however, a plurality of ultrasonic transmission units 241 and a plurality of ultrasonic reception units 242 may be disposed in other modes.

FIG. 3 schematically shows a perspective view of the inside of the ultrasonic inspection apparatus 1 seen from the side with the arrow A in FIG. 1A. The ultrasonic inspection apparatus 1 includes, inside the housing 11, a printed circuit board P1 on which various electronic components are mounted, and a printed circuit board P2 on which the ultrasonic transmission units 241 and the ultrasonic reception units 242 are mounted. The printed circuit board P1 and the printed circuit board P2 are electrically connected to each other by a connector or cable (not shown).

The speaker 22 is provided on the side of the housing 11 of the ultrasonic inspection apparatus 1, which is opposite to the side on which the ultrasonic transmission units 241 and the ultrasonic reception units 242 are provided. The speaker 22 is electrically connected to the printed circuit board P1 by a connector or cable.

### [Functional Configuration of Ultrasonic Inspection Apparatus 1]

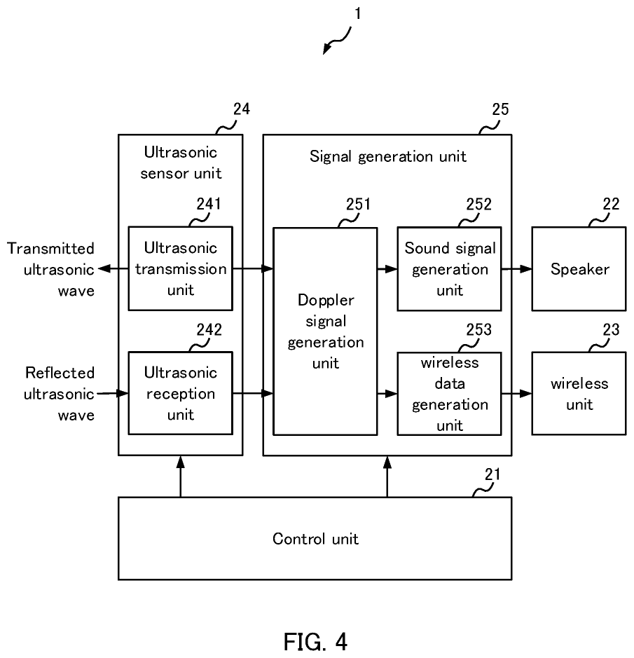

FIG. 4 is a block diagram showing a functional configuration of the ultrasonic inspection apparatus 1. As shown in FIG. 4, the ultrasonic inspection apparatus 1 includes a control unit 21, the speaker 22, a wireless unit 23, an ultrasonic sensor unit 24 and a signal generation unit 25. For example, the control unit 21, the wireless unit 23 and the signal generation unit 25 are mounted on the printed circuit board P1, and the ultrasonic sensor unit 24 is mounted on the printed circuit board P2. The signal generation unit 25 includes a Doppler signal generation unit 251, a sound signal generation unit 252 and a wireless data generation unit 253.

The control unit 21 includes, for example, a central processing unit (CPU), a read-only memory (ROM) and a random access memory (RAM), and the CPU controls the operation of the ultrasonic inspection apparatus 1 by executing a program stored in the ROM. The ultrasonic inspection apparatus 1 may execute at least part of the functions of the signal generation unit 25.

The speaker 22 outputs a sound signal corresponding to the Doppler signal. The speaker 22 is provided on the side opposite to the side where the ultrasonic sensor unit 24 is provided on the printed circuit board P1. By providing the speaker 22 on the side opposite to the side on which the ultrasonic sensor unit 24 is provided, howling is less likely to occur. Further, by providing the speaker 22 on the side opposite to the side on which the ultrasonic sensor unit 24 is provided, the heart rate can be sensed through, in addition to the ears, the vibration due to sound being transmitted to the user's hand.

The speaker 22 can output sound in a predetermined frequency range (i.e. resonance frequencies) more loudly than the sound at other frequencies. The lower limit of the resonant frequency range is the frequency at which the electrical impedance becomes the largest in the audible band. Although the resonance frequency range of the speaker 22 is arbitrary, the resonance frequency range of the speaker 22 of the present embodiment is, for example, from 200 Hz to 10 kHz, and the sound pressure level of the sound output by the speaker 22 can be −10 dB or more in the range from 200 Hz to 10 kHz.

Further, the speaker 22 is provided near the position of the area where a plurality of apertures 13 shown in FIG. 1A are provided. Since the speaker 22 is provided at a position between the center position of the ultrasonic inspection apparatus 1 and the outer periphery of the ultrasonic inspection apparatus 1, the user can easily hold the ultrasonic inspection apparatus 1 so that the speaker 22 is not blocked by the palm. For example, the user can easily hear the sound output from the speaker 22 by holding the ultrasonic inspection apparatus 1 such that the little finger is located on the side with the arrow A in FIG. 1A, thereby exposing at least some of the apertures 13 between the fingers.

The wireless unit 23 transmits data corresponding to the Doppler signal to the external terminal. The wireless unit 23 transmits data indicating, for example, a heart rate waveform. The wireless unit 23 transmits electric waves complying with, for example, Wi-Fi (registered trademark) or Bluetooth (registered trademark) standards.

The ultrasonic sensor unit 24 includes the ultrasonic transmission unit 241 for transmitting ultrasonic waves, and the ultrasonic reception unit 242 for receiving reflected ultrasonic waves, which are ultrasonic waves resulting from the transmitted ultrasonic waves transmitted from the ultrasonic transmission unit 241 being reflected within the human body. The ultrasonic transmission unit 241 transmits an ultrasonic wave of 1 MHz as a frequency suitable for, for example, monitoring the fetal heart rate. The ultrasonic transmission unit 241 starts or stops transmission of ultrasonic waves based on an instruction provided from the control unit 21 in accordance with an operation of the user (e.g., an operation of turning on a power source). The ultrasonic transmission unit 241 inputs a signal based on the transmitted ultrasonic waves to the Doppler signal generation unit 251. The ultrasonic reception unit 242 inputs a signal based on the received reflected ultrasonic waves to the Doppler signal generation unit 251.

The signal generation unit 25 generates various signals. Specifically, the Doppler signal generation unit 251 generates a Doppler signal based on the difference between the frequency of the transmitted ultrasonic wave and the frequency of the reflected ultrasonic wave. The Doppler signal generation unit 251 generates the Doppler signal on the basis of, for example, the autocorrelation function of the reflected ultrasonic wave by using a known autocorrelation method. The Doppler signal is, for example, a signal indicating a value corresponding to a flow velocity of the blood that reflects the transmitted ultrasonic wave. If the frequency of the ultrasonic wave transmitted by the ultrasonic transmission unit 241 is 1 MHz, the frequency of the Doppler signal is, for example, about 100 Hz. The Doppler signal generation unit 251 inputs the generated Doppler signal to the sound signal generation unit 252.

The sound signal generation unit 252 generates a sound signal corresponding to the Doppler signal. The sound signal generation unit 252 generates a sound signal having a magnitude corresponding to, for example, the intensity of the reflected ultrasonic wave or the intensity of the Doppler signal. The sound signal generation unit 252 inputs the generated sound signal to the speaker 22 and the sound based on the sound signal is output from the speaker 22. Since the sound signal generation unit 252 generates the sound signal having a magnitude corresponding to the intensity of the reflected ultrasonic waves or the intensity of the Doppler signal, the user can hear a loud heart rate sound if, for example, the ultrasonic wave is transmitted to a position close to the fetal heart. Therefore, the user can easily adjust the position of the ultrasonic inspection apparatus 1 based on the magnitude of the sound.

The sound signal generation unit 252 may generate a sound signal corresponding to the sum of the intensities of a plurality of reflected ultrasonic waves received by the plurality of ultrasonic reception units 242 or the sum of the intensities of a plurality of Doppler signals based on each of the plurality of reflected ultrasonic waves, so that the magnitude of the sound signal changes with high precision according to the position of the object to be inspected. The sum of the intensities of the plurality of reflected ultrasonic waves or the sum of the intensities of the plurality of Doppler signals is greater when the intensities of the reflected ultrasonic waves detected by each of the plurality of ultrasonic reception units 242 are at an equivalent level than when there is a large difference between the intensities of the reflected ultrasonic waves detected by some ultrasonic reception units 242 among the plurality of the ultrasonic reception units 242 and the intensities of the reflected ultrasonic waves detected by other ultrasonic reception units 242.

Therefore, as the position of the object to be inspected becomes closer to the center position of the ultrasonic inspection apparatus 1 and the variation between the intensities of the reflected ultrasonic waves detected by the plurality of ultrasonic reception units 242 becomes smaller, the sum of the intensities of the plurality of reflected ultrasonic waves or the intensities of the plurality of Doppler signals becomes greater, and the sound speaker 22 therefore emits a louder sound. As a consequence, the user can easily adjust the center position of the ultrasonic inspection apparatus 1 to a desired position (for example, a position close to the fetal heart), while listening to the sound emitted by the speaker 22.

Incidentally, in human auditory characteristics, the sensitivity to sound of frequencies equal to or less than 200 Hz is relatively low. Therefore, if the frequency of the Doppler signal is about 100 Hz and if the Doppler signal of 100 Hz is output as-is from the speaker 22, it is difficult for the user to hear the Doppler signal. Therefore, the sound signal generation unit 252 generates the sound signal by, for example, multiplying the Doppler signal. Specifically, the sound signal generation unit 252 multiplies the Doppler signal so that the sound signal is included in the frequency range including the resonance frequency of the speaker.

More specifically, the sound signal generation unit 252 generates the sound signal by multiplying the Doppler signal by a factor of two or more. If the frequency of the Doppler signal is about 100 Hz, then the speaker 22 can generate a sound signal having a frequency of about 200 Hz or more by having the sound signal generation unit 252 multiply the Doppler signal by a factor of two or more. Therefore, the user can easily hear the heart rate sound that is based on the Doppler signal. In particular, if the resonance frequency range of the speaker 22 is between 200 Hz and 10 kHz, it is preferred that the sound signal generation unit 252 multiplies the Doppler signal by a factor of two or three.

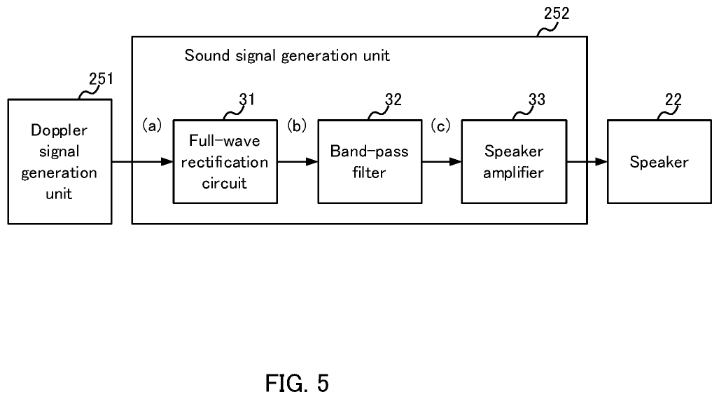

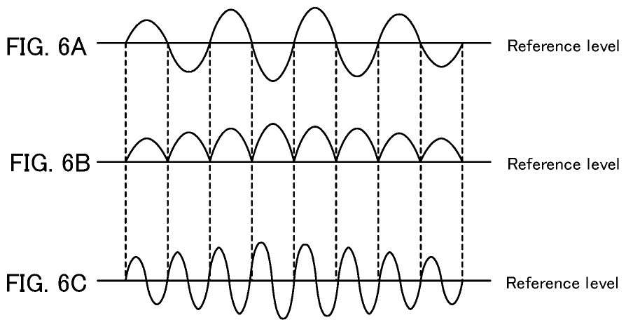

FIG. 5 is a diagram showing a configuration example of the sound signal generation unit 252. FIG. 6 is a diagram for explaining the multiplication process in the sound signal generation unit 252. As shown in FIG. 5, the sound signal generation unit 252 includes a full-wave rectification circuit 31, a band-pass filter 32 and a speaker amplifier 33.

FIG. 6A shows an exemplary waveform of the Doppler signal input from the Doppler signal generation unit 251 to the full-wave rectification circuit 31. FIG. 6B shows an exemplary waveform of the signal output from the full-wave rectification circuit 31. FIG. 6C shows an exemplary waveform of the signal output from the band-pass filter 32. It should be noted that the waveforms shown in FIG. 6A to 6C are schematic waveforms and are different from the actual waveforms.

By subjecting the Doppler signal input from the Doppler signal generation unit 251 to full-wave rectification, the full-wave rectification circuit 31 converts the signal below the reference level to a signal equal to or above the reference level. The full-wave rectification circuit 31 inputs the converted signal to the band-pass filter 32.

The bandpass filter 32 is a filter that passes, among the frequency components included in the signal input from the full-wave rectification circuit 31, a predetermined frequency component included in the audible range of the user; and does not pass other frequency components. The band-pass filter 32 passes the frequency component between, for example, 200 Hz and 10 kHz, to the speaker amplifier 33. By extracting the frequency component between 200 Hz and 10 kHz from the signal shown in FIG. 6B, a signal obtained by multiplying the Doppler signal shown in FIG. 6A by a factor of two is generated as shown in FIG. 6C.

The occurrence of howling due to the vibration of the ultrasonic sensor unit 24 caused by the vibration emitted by the speaker 22 is also prevented by the speaker 22 outputting the sound having the frequency obtained by multiplying the frequency of the Doppler signal. It should be noted that, in order to prevent the occurrence of howling, the sound signal generation unit 252 may limit the maximum value of the sound signal to a magnitude where howling does not occur if the intensity of the reflected ultrasonic wave is at a maximum. For example, the speaker 22 generates a sound signal having a magnitude corresponding to the intensity of the reflected ultrasonic wave if the intensity of the reflected ultrasonic wave is less than a threshold value, and it generates a sound signal corresponding to the intensity of the reflected ultrasonic wave equal to the threshold if the intensity of the reflected ultrasonic wave is equal to or greater than the threshold value.

### [Separation of Fetal and Maternal Heart Sounds]

When the user inspects the fetal heart rate using the ultrasonic inspection apparatus 1, the reflected ultrasonic waves received by the ultrasonic reception units 242 include a signal synchronized with the fetal heart rate and a signal synchronized with the heart rate of the mother. The sound signal generation unit 252 selectively generates a sound signal corresponding to the fetal heart rate in order to make it easier for the user to hear the fetal heart rate sound. Specifically, the reflected ultrasonic waves includes a first reflected ultrasonic wave reflected from the fetus in the human body and a second reflected ultrasonic wave reflected from the mother's body, and the sound signal generation unit 252 generates a sound signal corresponding to the Doppler signal generated by the Doppler signal generation unit 251 based on the first reflected ultrasonic wave.

Among the sound signals generated based on the Doppler signals, the sound signal generation unit 252 generates a sound signal to be output to the speaker 22 by, for example, removing a frequency component corresponding to a cycle within a predetermined range with respect to the mother's average heart rate cycle (for example, a cycle within a range not including the fetus's average heart rate cycle) and by extracting a frequency component corresponding to a cycle within a predetermined range with respect to the fetus's average heart rate cycle. The sound signal generation unit 252 may generate a sound signal to be output to the speaker 22 by making the amplification factor for the frequency component corresponding to the fetus's average heart rate cycle larger than the amplification factor for the frequency component corresponding to the mother's average heart rate cycle.

### [Wireless Transmission of Heart Rate Signal]

The wireless data generation unit 253 generates wireless data for the wireless unit 23 to transmit to an external terminal on the basis of the Doppler signal. The wireless data generation unit 253 converts, for example, the Doppler signal into a digital signal and generates the wireless data having a frame format defined by a predetermined wireless communication protocol. The wireless data generation unit 253 inputs the generated wireless data to the wireless unit 23.

### [Effects of Ultrasonic Inspection Apparatus 1 According to First Embodiment]

As described above, the ultrasonic inspection apparatus 1 emits, with the speaker 22, a sound signal corresponding to the Doppler signal based on the difference between the frequency of the transmitted ultrasonic wave and the frequency of the reflected ultrasonic wave. Due to the ultrasonic inspection apparatus 1 emitting the sound signal in this manner, the user can hear the sound indicating the state of the region to be inspected while performing the inspection using the ultrasonic inspection apparatus 1. Accordingly, for example, when the user seeks to check the fetal state, the user can hear the fetal heart rate sound while watching the mother's body which is in contact with the ultrasonic inspection apparatus 1, and the user can therefore easily search for the optimum position and operability is thereby improved.

In particular, the ultrasonic inspection apparatus 1 can emit sound, the magnitude of which changes according to the intensity of the reflected ultrasonic wave or the intensity of the Doppler signal based on the reflected ultrasonic wave, and thus, a louder sound is emitted when the ultrasonic inspection apparatus 1 is closer to the position of the fetus. Accordingly, the user can search for the optimum position by referring to the loudness of the sound.

Further, the ultrasonic inspection apparatus 1 generates a sound signal by multiplying the Doppler signal so that sound having a frequency matching the resonance frequency of the speaker 22 can be emitted. This configuration of the ultrasonic inspection apparatus 1 enables the user to easily hear the fetal heart rate sound.

### Second Embodiment

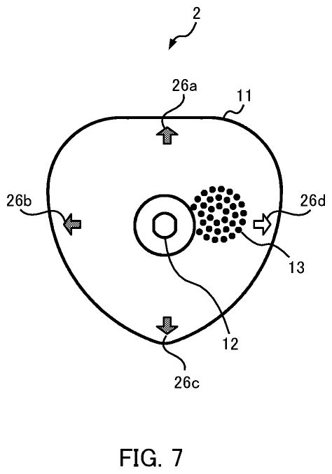

FIG. 7 is a diagram showing an external appearance of an ultrasonic inspection apparatus 2 according to the second embodiment. The ultrasonic inspection apparatus 2 differs from the ultrasonic inspection apparatus 1 shown in FIG. 1A to 1C in that it further includes a display unit 26 on the side on which the protrusion 12 is provided, i.e. on the side opposite to the side on which the ultrasonic transmission units 241 and the ultrasonic reception units 242 are provided, but it is equivalent to the ultrasonic inspection apparatus 1 in other respects. The display unit 26 displays information indicating the intensity of the reflected ultrasonic waves received by the ultrasonic reception units 242. The display unit 26 is provided on the side of the housing 11 of the ultrasonic inspection apparatus 1 opposite to the side on which the ultrasonic sensor unit 24 is provided.

The display unit 26 includes, for example, a plurality of light-emitting elements provided at least at four positions outward from the center position of the housing 11 of the ultrasonic inspection apparatus 2. In the example shown in FIG. 7, display units 26a, 26b, 26c, 26d, which are light-emitting areas in the form of an arrow, are respectively provided on the back side, the left side, the front side, and the right side with respect to the center position of the housing 11, as the plurality of light-emitting elements. The brightness of each of the display units 26a, 26b, 26c, 26d changes in synchronization with the change in the intensity of the sound signal. For example, the larger the intensity of the sound signal, the brighter each of the display units 26a, 26b, 26c, 26d is.

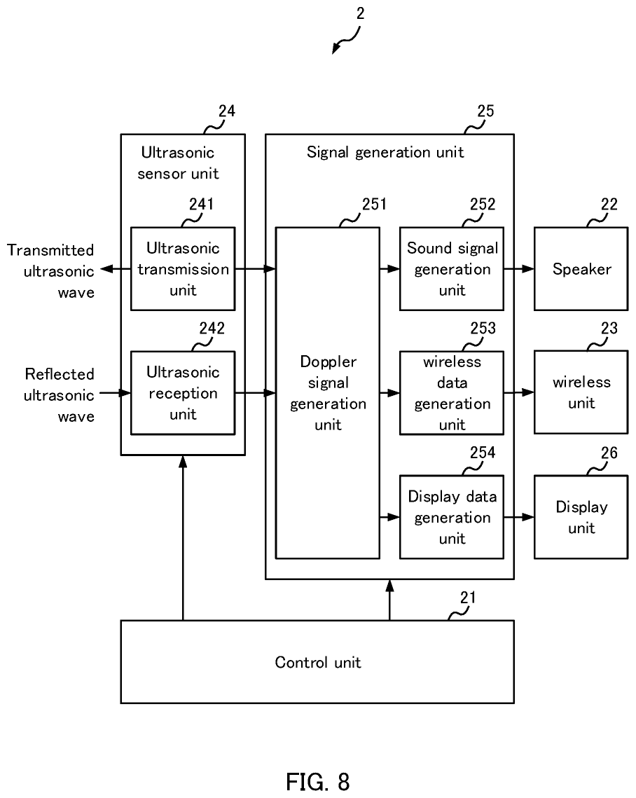

FIG. 8 is a block diagram showing a functional configuration of the ultrasonic inspection apparatus 2. The block diagram shown in FIG. 8 differs from the block diagram of the ultrasonic inspection apparatus 1 shown in FIG. 4 in that the signal generation unit 25 further includes a display data generation unit 254.

The display data generation unit 254 generates display data based on the reflected ultrasonic waves. The display data generation unit 254 generates, for example, display data showing a size corresponding to the intensity of the reflected ultrasonic waves. The display data generation unit 254 inputs the generated display data to the display unit 26, and the display unit 26 displays information corresponding to the display data. The display data generation unit 254 may also generate display data based on the Doppler signals.

The display data generation unit 254 generates, for example, display data for displaying information corresponding to the intensity of the Doppler signals on the display unit 26. Specifically, the display data generation unit 254 generates display data corresponding to higher brightness as the intensity of the Doppler signals becomes larger. With this configuration of the display data generation unit 254, the closer the ultrasonic inspection apparatus 2 is to, for example, the position of the fetus, the brighter the display shown by the display unit 26. As a consequence, the user can easily determine whether or not the ultrasonic inspection apparatus 2 is close to the position of the fetus.

The display data generation unit 254 may generate display data with which the size changes according to a change in the intensity of the Doppler signals. In this case, as the brightness of the display unit 26 changes in synchronization with, for example, a fetal heart rate, the user can easily grasp the state of the heart rate.

The Doppler signal generation unit 251 may generate a plurality of Doppler signals based on the difference between the frequencies of the transmitted ultrasonic waves and the frequencies of the reflected ultrasonic waves received by each of the plurality of ultrasonic reception units, and the display data generation unit 254 may generate display data corresponding to the sum of the intensities of the plurality of Doppler signals. At this time, the display data generation unit 254 may generate the display data based on the intensities of the plurality of Doppler signals corresponding to the fetal heart rate.

For example, the display data generation unit 254 generates display data corresponding to the sum of the intensities of a plurality of reflected ultrasonic waves received by the plurality of ultrasonic reception units 242, or the sum of the intensities of a plurality of Doppler signals based on the plurality of reflected ultrasonic waves. The sum of the intensities of the plurality of reflected ultrasonic waves or the sum of the intensities of the plurality of Doppler signals is greater when the intensities of the reflected ultrasonic waves detected by each of the plurality of ultrasonic reception units 242 are at an equal level than when there is a large difference between the intensities of the reflected ultrasonic waves detected by some ultrasonic reception units 242 among the plurality of the ultrasonic reception units 242 and the intensities of the reflected ultrasonic waves detected by other ultrasonic reception units 242.

Therefore, as the position of the object to be inspected becomes closer to the center position of the ultrasonic inspection apparatus 2 and the variation between the intensities of the reflected ultrasonic waves detected by the plurality of ultrasonic reception units 242 becomes smaller, the sum of the intensities of the plurality of reflected ultrasonic waves or the intensities of the plurality of Doppler signals becomes greater, and the display unit 26 emits brighter light. As a consequence, the user can easily adjust the center position of the ultrasonic inspection apparatus 2 to a desired position (for example, a position close to the fetal heart), while visually recognizing the light emitted from the display unit 26.

The display data generation unit 254 may generate display data indicating an orientation identified based on the intensities of the reflected ultrasonic waves received by each of the plurality of ultrasonic reception units 242. The display data generation unit 254 generates, for example, display data for making the display unit 26 brighter, which is provided at a position corresponding to the ultrasonic reception unit 242 that received a relatively strong reflected ultrasonic wave, from among the plurality of ultrasonic reception units 242.

The display data generation unit 254 may generate display data indicating an orientation identified on the basis of the intensities of the Doppler signals based on the reflected ultrasonic waves received by each of the plurality of ultrasonic reception units 242. The display data generation unit 254 generates, for example, display data for making the display unit 26 brighter, which is provided at a position corresponding to the ultrasonic reception unit 242, in which relatively strong Doppler signals are generated, from among the plurality of ultrasonic reception units 242. At this time, the display data generation unit 254 may generate the display data based on the intensities of the plurality of Doppler signals corresponding to the fetal heart rate.

For example, the display data generation unit 254 generates display data for recommending moving the ultrasonic inspection apparatus 2 to a position where the ultrasonic reception unit 242 which received a relatively large-intensity reflected ultrasonic wave from among the plurality of ultrasonic reception units 242 is provided. Specifically, if the intensity of the Doppler signal based on the reflected ultrasonic waves received by the ultrasonic reception units 242 disposed on the right side with respect to the center position of the ultrasonic inspection apparatus 2 is relatively large, the display data generation unit 254 makes the display unit 26 provided on the right side (the display unit 26d in the example shown in FIG. 7) of the ultrasonic inspection apparatus 2 brighter than the other display units 26. The display data generation unit 254 may generate display data causing the display unit 26 to blink on the side to which the ultrasonic inspection apparatus 2 is to be moved.

This configuration of the display data generation unit 254 enables the user to grasp the side (e.g., the back side, the left side, the front side, or the right side), with respect to the ultrasonic inspection apparatus 2, on which the position where the fetal heart rate is strongly detected is located. Therefore, the user can easily move the ultrasonic inspection apparatus 2 to a position where the fetal heart rate can be easily detected.

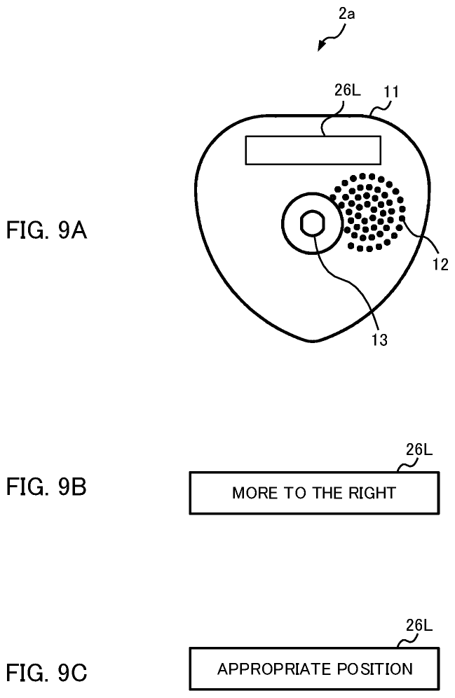

FIG. 9 is a diagram showing an external appearance of an ultrasonic inspection apparatus 2a, which is a variation example of the ultrasonic inspection apparatus 2 according to the second embodiment. The ultrasonic inspection apparatus 2a differs from the ultrasonic inspection apparatus 2 in that a display unit 26L is provided instead of the display unit 26 in the ultrasonic inspection apparatus 2 shown in FIG. 7. The display unit 26L is, for example, a liquid crystal display and can display text or images based on the display data generated by the display data generation unit 254. Specifically, based on the intensities of the reflected ultrasonic waves received by the ultrasonic reception units 242 or on the intensities of the Doppler signals generated by the Doppler signal generation unit 251, the display data generation unit 254 generates display data including information for assisting in moving the ultrasonic inspection apparatus 2a so that the intensities of the reflected ultrasonic waves or the Doppler signals increase.

FIG. 9B and FIG. 9C show examples of the text displayed on the display unit 26L. FIG. 9B illustrates a case where the display unit 26L displays “MORE TO THE RIGHT” as a text displayed if the intensity of the reflected ultrasonic waves received by the ultrasonic reception units 242 disposed on the right side of the ultrasonic inspection apparatus 2a or the Doppler signals based on such reflected ultrasonic waves is relatively large. A user who visually recognizes this text can move the ultrasonic inspection apparatus 2a to the right to bring the center position of the ultrasonic inspection apparatus 2a closer to the position of the object to be inspected.

FIG. 9C illustrates a case where “APPROPRIATE POSITION” is displayed on the display unit 26L as a text displayed if the position of the ultrasonic inspection apparatus 2a is appropriate. If, for example, the intensities of the plurality of reflected ultrasonic waves received by each of the plurality of ultrasonic reception units 242 are equal to or greater than a predetermined value and if the variation in the intensities of the plurality of reflected ultrasonic waves is within a predetermined range, the display data generation unit 254 generates display data indicating that the position of the ultrasonic inspection apparatus 2a is appropriate. The display data generation unit 254 may also generate display data indicating that the position of the ultrasonic inspection apparatus 2a is appropriate if the intensities of the plurality of Doppler signals based on the reflected ultrasonic waves received by each of the plurality of ultrasonic reception units 242 are equal to or greater than a predetermined value and if the variation in the intensities of the plurality of Doppler signals is within a predetermined range. This configuration of the display data generation unit 254 enables the user to easily maintain the position of the ultrasonic inspection apparatus 2a if the position of the ultrasonic inspection apparatus 2a is appropriate.

### [Effects of Ultrasonic Inspection Apparatus 2 According to Second Embodiment]

As described above, the ultrasonic inspection apparatus 2 displays information corresponding to the display data generated based on the reflected ultrasonic waves. The ultrasonic inspection apparatus 2 causes, for example, light-emitting elements to emit light at a brightness corresponding to the intensity of the reflected ultrasonic waves or to the intensity of the Doppler signals based on the reflected ultrasonic waves, or causes a display to display information indicating content corresponding to the intensity. This configuration of the ultrasonic inspection apparatus 2 enables the user to grasp whether or not the position of the ultrasonic inspection apparatus 2 is appropriate while operating the ultrasonic inspection apparatus 2, and operability is thereby improved. In particular, the ultrasonic inspection apparatus 2 can display the information indicating the orientation of the appropriate position, and therefore, if the user notices that the fetus has not been successfully measured due to the fetus moving inside the womb after starting the measurement with the ultrasonic inspection apparatus 2 at a fixed position, the ultrasonic inspection apparatus 2 can be moved to the appropriate orientation.

### Third Embodiment

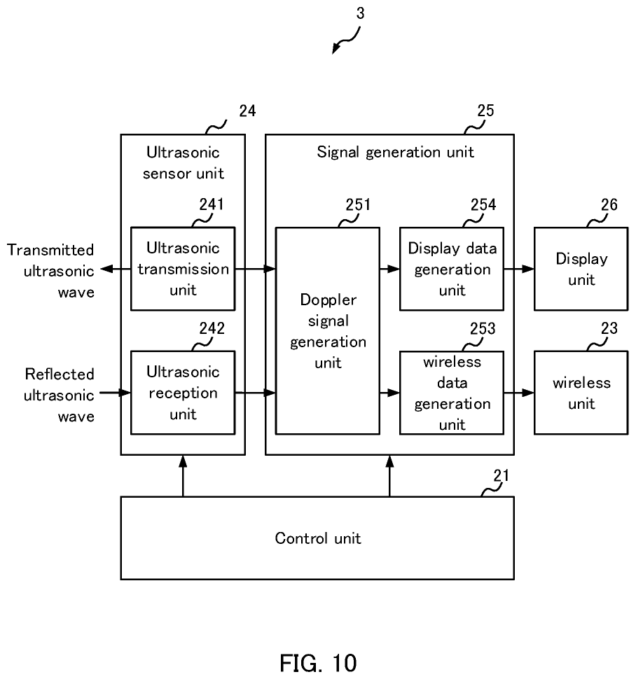

FIG. 10 is a block diagram showing a functional configuration of an ultrasonic inspection apparatus 3 according to the third embodiment. The ultrasonic inspection apparatus 3 differs from the ultrasonic inspection apparatus 2 shown in FIG. 8 in that the ultrasonic inspection apparatus 3 does not include a sound signal generation unit 252, but it is equivalent to the ultrasonic inspection 2 in other respects. The ultrasonic inspection apparatus 3 outputs the display data based on the reflected ultrasonic waves or the Doppler signals, without outputting the sound signal. The contents of the display data generated by the display data generation unit 254 are similar to those of the ultrasonic inspection apparatus 2 according to the second embodiment. Even if the ultrasonic inspection apparatus 3 does not output a sound signal, the user can still recognize how to move the ultrasonic inspection apparatus 3 based on the displayed information, and operability is thereby improved.

The present invention is described using the embodiments of the present invention. The technical scope of the present invention is, however, not limited to the scope described in the above embodiments and various variations and modifications are possible within the scope of the gist thereof. For example, the specific embodiments of the distribution and integration of the apparatuses are not limited to the above embodiments, and all or part thereof can be configured with any unit in a functionally or physically dispersed or integrated manner. Further, new embodiments generated by arbitrary combinations of a plurality of embodiments are also included in the embodiments of the present invention. Further, the effects of the new embodiments brought by the combinations also have the effects of the original embodiments.

What is claimed is:

1. An ultrasonic inspection apparatus comprising:
an ultrasonic sensor unit including an ultrasonic transmission unit for transmitting an ultrasonic wave and an ultrasonic reception unit for receiving a reflected ultrasonic wave, the reflected ultrasonic wave being an ultrasonic wave resulting from the transmitted ultrasonic wave that is transmitted by the ultrasonic transmission unit and reflected in a human body;
a Doppler signal generation unit for generating a Doppler signal based on a difference between a frequency of the transmitted ultrasonic wave and a frequency of the reflected ultrasonic wave;
a sound signal generation unit for generating a sound signal corresponding to the Doppler signal; and
a speaker for outputting the sound signal.

2. The ultrasonic inspection apparatus according to claim 1, wherein
the speaker is provided on a side of a housing of the ultrasonic inspection apparatus opposite to a side on which the ultrasonic sensor unit is provided.

3. The ultrasonic inspection apparatus according to claim 1, further comprising:
a printed circuit board on which the Doppler signal generation unit and the sound signal generation unit are provided, wherein
the speaker is provided on a side opposite to a side where the ultrasonic sensor unit is provided on the printed circuit board.

4. The ultrasonic inspection apparatus according to claim 3, further comprising:
an integrated housing that accommodates at least the ultrasonic sensor unit, the printed circuit board, and the speaker, wherein
the speaker is fixed so that the relative position with respect to the ultrasonic sensor unit does not change on the side of the housing opposite to the side on which the ultrasonic sensor unit is provided on the printed circuit board.

5. The ultrasonic inspection apparatus according to claim 1, wherein
the sound signal generation unit generates the sound signal by multiplying the Doppler signal.

6. The ultrasonic inspection apparatus according to claim 5, wherein
the sound signal generation unit multiplies the Doppler signal such that the sound signal is included in a frequency range that includes a resonance frequency of the speaker.

7. The ultrasonic inspection apparatus according to claim 5, wherein
the sound signal generation unit generates the sound signal by multiplying the Doppler signal by a factor of two or more.

8. The ultrasonic inspection apparatus according to claim 1, wherein
the sound signal generation unit generates the sound signal having a magnitude corresponding to an intensity of the reflected ultrasonic wave.

9. The ultrasonic inspection apparatus according to claim 8, wherein
the ultrasonic sensor unit includes a plurality of the ultrasonic reception units, and
the sound signal generation unit generates the sound signal corresponding to a sum of intensities of a plurality of the reflected ultrasonic waves received by the plurality of ultrasonic reception units.

10. The ultrasonic inspection apparatus according to claim 8, wherein
the sound signal generation unit limits the maximum value of the sound signal to a magnitude at which howling does not occur if the intensity of the reflected ultrasonic wave is at a maximum.

11. The ultrasonic inspection apparatus according to claim 1, wherein
the sound signal generation unit generates the sound signal corresponding to the Doppler signal generated by the Doppler signal generation unit on the basis of a first reflected ultrasonic wave reflected from a fetus in the human body among the first reflected ultrasonic wave and a second reflected ultrasonic wave reflected from a mother's body corresponding to the human body.

12. The ultrasonic inspection apparatus according to claim 1, further comprising:
a display unit provided on a side of the housing of the ultrasonic inspection apparatus opposite to a side on which the ultrasonic sensor unit is provided, the display unit displaying information indicating the intensity of the reflected ultrasonic wave received by the ultrasonic reception unit.

13. The ultrasonic inspection apparatus according to claim 12, wherein
the display unit includes a light-emitting element, and brightness of the light-emitting element changes in synchronization with a change in the intensity of the sound signal.
